SEQUENCE LISTING (3 sequences)

SEQ ID NO: 1 - DNA, 24 bp, Artificial
  Forward primer Tert-1-F
cagcgtcaggaaataaatgacaca

SEQ ID NO: 2 - DNA, 28 bp, Artificial
  Reverse primer Tert-1-R
ataacaacagaagacagaggctactttg

SEQ ID NO: 3 - DNA, 24 bp, Artificial
  Dual-labeled flourescent probe Tert-1-Probe
ncctgcctccagcttcctggccan

(19) United States
(12) Patent Application Publication
(10) Pub. No.: US 2005/0164252 A1
(43) Pub. Date: Jul. 28, 2005

(54) Methods using non-genic sequences for the detection, modification and treatment of any disease or improvement of functions of a cell

(76) Inventor: Wah Hin Alex Yeung, Hong Kong SAR (HK)

Correspondence Address: Plasmagene Limited;5/F, Club Lusinato, 16 Ice House Street, Central, Hong Kong SAR, HK

(21) Appl. No.: 10/997,890

(22) Filed: Nov. 29, 2004

Related U.S. Application Data
(60) Provisional application No. 60/526,597, filed on Dec. 4, 2003.

(51) Int. Cl. C12Q 1/68; A01H 1/02; C12N 15/82

(52) U.S. Cl. 435/6; 514/44; 800/293

(57) ABSTRACT

This invention provides the use of conserved non-genic sequences so commonly found in most species of plants and animals for the detection of a disease and condition. The intimate and ultimately important link of the corresponding DNA sequences and expressed RNA sequences with their conserved non-genic sequence makes the detection possible. Apart from the diagnostic use, the combination of the conserved non-genic sequences with the corrected or designed DNA or RNA sequences makes treatment or improvement possible for living organisms.

### BACKGROUND OF THE INVENTION

It has long been assumed that only 5% of the human genome contains useful information for the development and function of the human body and those sequences are referred to as genes. The rest has been neglected in the past and referred to as non-genic sequences or junk genes with assumingly no role in human genetics. These non-genic sequences are considered quite useless for a long time. There are some definitions and classifications based on their behaviour in a small portion of the genome of these so called junk sequences. Apart from the proper coding genes with the right sequence identity to human cDNAs and EST (expressed sequences tag), there are partial genes which are possibly pre-pseudogenes with intact ORFs, non-coding RNA genes which are further subdivided into small and micro RNA genes, genes with no ORF and potential antisense sequences, protein coding genes and pseudogenes. Little is known about the other non-genic or unclassifiable sequences present in the vast majority of the rest of the genome.

In an article published in Science¹, highly conserved non-genic sequences are present in most placental mammals and can be easily distinguishable from transcribed sequences as well as protein coding sequences and non coding RNA genes. They are not likely to code for proteins, but may be protein binding. They are different from transcription factor binding sites, which are short and are embedded in less conserved sequences. These highly conserved non-genic sequences, are estimated to be subjected to a very strong and continual selective constraint, enabling them to remain largely unchanged for many million of years. A descriptive term of conserved non-genic sequences (CNGs) is used hereon.

One of the possible reasons suggested by this invention is that these CNGs are useful as guiding vehicles in cell differentiation and evolution process when disintegrated cells release all of their genetic materials into the blood stream after cell death, from either necrosis or apoptosis. Since most of the genetic materials such as DNAs and RNAs will undergo rapid fragmentation or degradation except those that are being bound and protected by protein molecules², important information of the cellular conditions in progress before cell death would have been lost. The hypothesis of this invention is that the highly conserved non-genic sequences of a particular chromosome or region will be preserved with the appropriate disintegrating DNAs and RNAs (collectively named as diDRNAs) close to that chromosome or region by protein molecules that can withstand degradation until they are destined to reach and affect another target, most likely another cell. Once the target is reached, and especially if the cell is dividing or going to divide, the CNGs will guide the companion DNAs and RNAs and home in onto the corresponding and likely complimentary CNGs (located in the same chromosome number) for interactions, very much like the RISC protein (RNA Interference Silencing Complex) in small interfering RNA silencing.

Such interactions can be complex. The most likely scenarios are that once the CNGs are matched up as described, the accompanied diDRNAs will likely be injected and accepted to replace the existing corresponding DNA or, in the case of RNA, follows the discovery of a RNA interference pathway affecting the genome as described by Schramke and Allshire³. Once the interaction is completed, the effect of the CNGs would likely be one of at least three scenarios. The first effect is a transient change of cell function if an adult cell is affected by the CNGs and diDRNAs. The likelihood will be further stimulation of the same population or different populations of adult cells to expand, divide, and undergo apoptotic changes in similar cycles of event as the first CNGs and diDRNAs complex until such time that the condition or disease set forth in the first cycle is either being overcome, stabilized or the host be overwhelmed. The expansion of these CNGs and diDRNAs can be detected below for the diagnosis of a disease (such as the examples illustrated below for trisomy 21 or cancer). The second scenario is based on the effects of the CNGs and diDRNAs on different stem cell populations of the host leading to genetic and epigenetic changes in a clone of cells. These changes of the different stem cells of the host may lead to successful overcoming of a disease or condition (e.g. increase hair or fur production to keep the host warm or the squamous metamorphosis in bronchial cells in smoking individuals to acclimate to smoke inhalation). It may also directly or indirectly leads to disease conditions such as cancer (e.g. the methylation of tumour suppressor gene p16 in a variety of cancers). The third scenario is determined by the incorporation of these CNGs and diDRNAs complexes with germ cell, from which the genetic and epigenetic changes will be passed on to the next generation. This can be illustrated by the rather rapid selection and “evolutional” changes in the development of sickle cell for malaria infection and lactose tolerance in milk allergy before these conditions or diseases can overwhelm a particular population.

This invention specifically points out to the usage of a joined complex or a separate mixture or concoction of CNGs with either DNAs, RNAs, or both. The hypothesis recognizes the constant interaction of all the elements so that genetic and epigenetic expression levels and mutation changes are always at play. The necessity, or the major function of the CNGs is to make sure the selection and possibly evolutional changes are taking place at the right spot, with the right insertion point and the right chromosome.

If this hypothesis can be projected even more in a forward fashion, e.g. the environmental or evolutional need for a set of organ or body part may be effectively induced by the “creation” of another “hog” type gene to accommodate the excessive demand from a large and specific influx of CNGs and DRNAs for that particular body part or organ.

This invention is to summarize the above hypothesis of these CNGs so that methods can be deduced that would be able to detect, modify, or treat cell genome particularly related to its functions and diseases.

### DESCRIPTION OF THE SPECIFIC EMBODIMENTS

Detection and Disease Diagnosis:

Detection of Diseases in the Fetus:

The presence of DNA in blood plasma or serum in the form of genetic sequences from different individuals has been well known. In the case of an allogeneic bone marrow transplantation, the reconstituted hemopoietic system of the recipient will consist of varying amount of the donor and the recipient's cells with corresponding differences in their genic sequences such as gender⁴ and DNA polymorphisms⁵. The approach from the above observation is that the analysed region has to bear a genetic difference in order that the analysis can yield a meaningful result. This result will then be used as a way to estimate the presence of a condition or disease. This observation then extends into tools used for prenatal diagnosis.

In this situation, the need to measure the genetic difference of different individuals has first been demonstrated by the detection of fetal DNA in maternal plasma and serum.⁶ Such a measurement may then be used for non-invasive prenatal diagnosis⁷⁻¹¹. It was apparent, from these publications, that relatively high absolute and relative concentrations of circulating fetal DNA in the maternal plasma and serum would be able to detect diseases such as fetal RhD incompatibility⁷, myotonic dystrophy⁹, achondroplasia¹¹ and other chromosomal translocations¹⁰. The principle behind the diagnosis of all these diseases is to find the genetic difference of the fetus that is inherited from the father's side only.

Besides the use of genetic differences, epigenetic differences such as methylation patterns of certain genes can also be used to differentiate individuals. This has been demonstrated in a recent patent application by Lo and Poon with methylated genes on the inactivated X chromosome as well as the IGF2-H19 locus¹².

Alternatively, the quantitative measurement of genetic materials in the form of genetic DNA, or mRNA derived mainly or exclusively from a different individual may also be used to detect disease conditions in prenatal non-invasive diagnosis using the maternal plasma or serum. The presence of such genetic DNA or mRNA, if contributed mainly by the fetus, may be indicative of certain disease conditions. The significant increase of the SRY DNA in the maternal serum of a male fetus is a good indicator of the presence of Down's syndrome whereas the increase of corticotropin releasing hormone (CRH) MRNA in the maternal plasma is a good measurement of pre-eclampsia¹³.

In this invention, the utilization of the absolute quantity and quality of non-genic sequences may be used as a measurement of disease conditions that affect the release of such sequences as well as differentiating the individuals in a sample specimen. This invention will demonstrate that the detection of non-genic sequences, which is not part of the genetic or epigenetic changes or characteristics, can also be used in the claims described.

In the first aspect, the present invention features methods so that detection of the quantity of non-genic sequences (NGs) which may be highly conserved (CNGs) can be used to detect fetal conditions from the maternal plasma or serum.

As described before, fetal DNA can be recovered from the maternal plasma in a significant quantity. The presence of conserved non-genic sequences which are particular linked to a special chromosome can theoretically be recovered with ease.

One of the most important fetal diseases is Trisomy 21 or Down's syndrome. It is highly likely that large amount of DNA materials from the fetus will be present in the maternal blood stream due to rapid necrosis or death of cells in the fetal placenta. Such a condition has been described in the previous publication using SRY as an indicator for measurement. The problem of SRY is, of course, that it can only be applied to the male fetus. In the case of the highly conserved non-genic sequences (CNGs) from the chromosome 21, such a problem does not exist. In addition, there are obvious cellular difficulties in transcribing the right genetic signals from the trisomy 21 chromosomes, resulting to an increase of faulty transcribed copies of genes in that chromosome. This is accompanied by an increase of the CNGs close to or relevant to the transcribed chromosome 21 genes.

If the hypothesis is correct, then the increase of fetal cell death, together with the high conservation of the chromosome 21 CNGs (with a multiple factor of three in number) after cell degradation, will push a large amount of these non-genic sequences into the maternal blood stream, possibly stabled with protein molecules. The detection of a significant amount of these non-genic sequences, with or without the relevant chromosome 21 gene products such as the disintegrating DNAs and RNAs, compared to a normal pregnancy will enable a non-invasive and early diagnosis of the disease of Trisomy 21. Please see working

### EXAMPLE I

Maternal blood plasma during late first trimester (11^{th} to 13^{th} week of gestation) and early second trimester (14^{th} to 16^{th} week of gestation) is collected from a group of pregnant women, preferably with a high probability of Trisomy 21 conception among a few of them

DNA from a given plasma sample is extracted in the usual manner and this is added to a primers set for one of the highly conserved non-genic sequences (CNAs) of chromosome 21. Appropriate probe for the same region (e.g. TaqMan) and a master mix for amplification is done using a real time PCR machine such as the ABI 7900. The total quantity of CNAs for this particular sequence is then recorded for this group of women.

The quantities of the CNAs DNA from Trisomy 21 mothers are matched with a group of normal mothers with similar weeks of gestation and the ROC curve is plotted to uncover whether it is a significant finding to use quantitative CNAs from chromosome 21 as a diagnostic marker for Trisomy 21 for expected mothers in the late first trimester and early second trimester.

If the hypothesis that CNGs are the guiding sequences for genomic modifications and evolutional selection, trisomy 21 fetus will indeed break down a lot of fetal placental cells with these highly conserved sequences passing into the maternal blood stream. Significant higher amount of these CNGs in the mother's blood can then be used as a non-invasive diagnosis of trisomy 21. The same hypothesis can be extended to other trisomies.

Alternatively, the above example can also be interpreted with the addition of the quantity of gene products from chromosome 21, e.g., some of the special critical region genes DNA and their associated RNAs. The increase presence of these products, together with the assay for the quantity of the relevant CNGs described above, further strengthens the diagnosis of trisomy 21.

### EXAMPLE II

The use of CNGs in the fetus can further be extended to other diseases apart from trisomy 21 above related to the use of the quantity of the CNGs confined to chromosome 21 or other trisomies. For if the hypothesis of CNGs related to chromosome functions is correct, any diseases that would involve any gene functions, whether it is an abnormal expression either towards the high or low side, or whether it is involved in mutations of any kind, should be accompanied by the CNGs close to and within the same chromosome. The detection of unusually high or low numbers of the CNGs next to or close to the involved gene within the same chromosome will point towards diseases involving that particular gene.

An example is given in the prenatal detection of cystic fibrosis. A maternal blood sample is taken to test for the presence as well as quantity of the CNGs close to the mutation of the cystic fibrosis gene in chromosome 7. The abnormal quantities of the cystic fibrosis CNGs, alone or together with abnormal quantities of the cystic fibrosis gene mutation DNA, e.g., the delta-F508 in exon 10 of the cystic fibrosis gene or the relevant RNA, can diagnose the presence of cystic fibrosis in the fetus.

Detection of Other Diseases:

The above hypothesis to detect conditions or diseases originated from gene abnormalities and mutations in the fetus can also be applied to the adult. In cancer, there are frequent mutations or silencing of tumour suppressor genes. Due to the varieties of the different mutation sites and a complicated system involving a large number of such genes, it may be difficult to use any one method as a cancer screening or detection program. Since CNGs may be activated during such a process together with that particular gene or genes, it should be much easier to detect the CNGs as a diagnosis.

### EXAMPLE III

The p53 gene is a tumour suppressor gene and if a person inherits only one good functional copy of the p53 gene from their parents, they are much more prone to cancer and may develop different tumours in a variety of tissues in early adulthood. This condition is known as Li-Fraumeni syndrome. In addition, mutations in p53 are also found in most tumour types that are not related to inheritance. The p53 gene has been mapped to chromosome 17. The gene product of p53 will interact with another gene to produce p21 protein, which acts as a stop sign to a cell division stimulator kinase protein (cdk2). Without such a stop sign, cells may divide uncontrollably and form tumours.

A working example of using this invention is to find the most relevant CNGs close to the p53 gene in chromosome 17. The detection of abnormally high or low amount of this particular p53 CNGs in the plasma or serum of normal individuals signifies an overly active, inactive or abnormal p53 gene, which leads to a general cancer screening test.

The quantification of the p53 CNGs will be similar to the steps taken for the detection of the trisomy 21 CNGs or the cystic fibrosis CNGs. Special primer and probe sets are designed for the best related CNGs close to the p53 gene. The identification and quantity of the CNGs DNA is then measured by any real time PCR machines using fluorescent probes such as the TaqMan probe. The process can of course be done by similar PCR systems or methodology of amplification known to those skilled in the art.

The above invention is also applicable to detection of other diseases. It has long been known that diseases that originate from genetic or epigenetic changes as well as changes of genetic expression can be measured by the appropriate DNAs or RNAs from the disease site or from the blood plasma or serum. This invention enables the use of a very stable and conserved non-genic region selected close to the site of genetic or epigenetic changes as a marker for the disease. In diseases that may involve multiple sites of mutation or polymorphism, a single test is now possible. For gene expression diseases, some with different isoforms, the measurement of CNG specific or close to the genetic site is far superior to the measurement of one or more of these highly unstable RNAs. In conclusion, a simple and stable test is available for a whole variety of diseases with high and real time sensitivity than the disease protein counterpart, even if that is available.

Modification of Gene Function and Gene Therapy

In order to understand that these highly conserved non-genic sequences (CNGs) may be able to modify cell genome and thereby its functions, an analogy to a similar phenomenon in RNA is described.

In the recent years, RNA interference creates much excitement in the biological field. The first sign of such an interference was shown in 1990 when biologist Rich Jorgensen tried to turn purple petunias more purple. He inserted a second copy of the rate-limiting enzyme gene. But instead of purple, the petunia was found to be white. He called this paradoxical effect “co-suppression” but at that time nobody knew why adding more of a gene that promoted a special color turned that gene off instead. Five years later, an experiment using a single strand of control “sense” strand of RNA worked as well to suppress the intended gene than the “anti-sense” strand. In 1998 Andrew Fire and Craig Mello solved the mystery by demonstrating that double stranded RNA was the real silencing agent. They called this “RNA interference” and a new field was born¹⁴. The Tuschl group extended the technique to mammalian cells and has made RNA interference or RNAi what it is today¹⁵.

In mammals, double stranded RNA or dsRNA acts mainly through post transcriptional mechanism targeting mRNA for destruction and the mediators for this sequence specific target recognition is now known to be consisted of about 21 nucleotide small interfering RNA (siRNA). These small siRNAs are produced normally from a much longer dsRNA that occurs in a natural state by a reaction involving Dicer Rnase III. After these siRNAs are formed, they are again taken up by another ribonuclease protein called RNA-inducing silencing complex (RISC). The RISC protein ultimately unwinds the siRNA to form a single strand and this will guide the RISC complex towards cytoplasmic target MRNA degradation¹⁶. In certain species, siRNA RISC complexes may also be able to incorporate into sequence specific DNA through chromatin or other sites that can effectively change genetic expression of that gene. Transitive RNAi can also occur if these siRNA are complementary to other RNA of the same or different targets. In some organisms, RNA-dependent or directed RNA polymerase (RdRP) can also prime siRNA synthesis using the target mRNA as template. The target RNA is then inactivated by Dicer RNA cleavage rather than by RISC. Some of the effects of siRNA silencing the target mRNA may sometimes be able also to amplify and spread throughout the organism, even when triggered by only minute quantities of dsRNA. This effect, however, has not been observed in mammals so far.

The theory of RNA interference can be taken as an example of the hypothesis of how these CNGs are working to preserve or modify cell genome after cell death. It is postulated that upon cell death either through necrosis or apoptosis, these CNGs will be conserved and protected with the appropriate RNAs and DNAs by protein particles. These complex molecules, similar to the RISC protein described in the RNA interference model, will then travel to different cellular sites so that so the CNGs may be able to guide the appropriate DNAs and RNAs to its proper insertion locations and chromosomes in the new cell genome. The recognition of the CNGs by the new genome may, and some will enable the incorporation of the appropriate signals from these semi-degraded DNAs and RNAs from the old dead cell. If the new genome is able to divide, it may then take cue from these signals for instructions to modify genetic transcriptions, hopefully to the advantage of the organism as a whole. If this is a normal cell, the effect will be limited by the number of cell divisions. If the new genome is an immune cell, it may be able to incorporate the signals from the CNGs and diDRNAs complex into lineage that will fight against incoming infections. If the new genome is a special stem cell, it may differentiate into a clone of genetically or epigenetically modified cell with long lasting beneficial or detrimental effect to the organism. If the new genome happens to be a germ cell, the incorporation of these genetic signals guided by the CNGs protein complex will effect selection and evolutional changes.

To utilize the invention in gene modification, gene transfer technologies as mentioned in the following example as a generic outline how it can be applied to the more specific examples that followed.

Gene Transfer Technologies:

There are many ways to deliver DNA and special RNA to eukaryotic cells and the ways described served only as examples and should not be construed as representing the whole picture of an ever changing scientific phenomenon.
1. DEAE-Dextran-mediated transfection: an old remedy that may still be used from time to time. It is limited by its toxicity to short term assays only.
2. Calcium phosphate mediated transfection: used for both transient or stable transfections but the method is difficult to be optimized and reproducibility is low.
3. Liposomes and similar molecules: Cationic carriers include lipids, polymers mixed with macromolecules of genetic materials.
4. Nonliposomal formulations: Lipids such as DOPE and polycationic carrier such as polyethylenimine can also be incorporated into nonliposomal formulations with high success.
5. Activated dendrimers: Highly branched molecules in which each branch terminates in a charge group which condense and compact the genetic materials.
6. Viral delivery: a viral with the modified gene is transfected into a cell. The cell produces viral particles that are colleted and used to infect the cells of interest, carrying the introduced gene with them. Retroviral particles insert their nucleic acids into the host genome but the cells must be dividing. Lentiviruses can integrate into non-dividing cells and lentiviral systems are now being used frequently. Adenoviruses can also be used. They are episomal and the genetic materials do not integrate into the genome. The advantage of the adenovirus system is that it can transduce a wide variety of cell types, both quiescent and proliferating, and the episomes may remain in the cell indefinitely, often at high copy number. To construct the vector for delivery, the gene of interest is cloned into a plasmid with an eviscerated adenoviral genome, which is then delivered into a packaging cell, which provides the missing adenoviral genes in trans. Adeno-associated virus (AAV) is another popular way of using virus for gene therapy. AAV appears to be quite harmless and can also infect non-dividing cells and integrates into the host genome at a specific location.
7. Electroporation: an electric shock to make the cell membrane transiently porous for gene insertion. It is notoriously cytotoxic.
8. Gene-gun approach: shooting genes into tissues at high velocity.
9. Microinjection: for very precious cell such as single cell zygotes to create transgenic organisms.
   Gene Modification and Gene Therapy:

With the above mentioned gene transfer technologies, CNGs and diDRNAs complex will be able to be incorporated to a cell target. Depending on the cell target and the specific CNGs and specially designed DNAs or RNAs (collectively named as deDRNAs) complex that are being inserted, cell function modification can take place. The designed DNA or RNA sequences will likely be the wild type (if mutation is at fault for the disease) or the corrected type (for improvement over the existing gene). This modification will be useful for maintaining or changing the health of the animal, or in other cases, fend off existing diseases. Certain working examples are shown below.

### EXAMPLE IV

Treatment of cancer or other serious inherited diseases can be effected by the use of the CNG complex to alter, epigenetic control of tumour suppressor genes. DNA methylation is one of the most important regulators of gene activity and the control is turned off in certain tumour suppressor gene. DNA methylation occurs mostly on one of DNA's four base pairs, cytosine. The epigenetic changes can be inherited or acquired. It's involvement in Prader-Willi syndrome and Beckwith-Wiedemann syndrome (BWS) is well known. In BWS, the epigenetic changes of genes H19 and LIT1 is associated with predisposition to cancer. The infusion of stem cell incorporated with the specific CNG complex can be used to alter the epigenetic changes that have taken place and prevent further disease progression or damage. The transfer of the specific complex through a vector to the affected tissue or cancer can mount effective tumour suppressor activity for cancer or produce the right protein to eliminate the disease. The treatment using the invention can be given in all of the usual channels known to those skilled in the art, such as intravenous infusion, intramuscular injection or the use of different vectors etc.

The construction of the CNG complex is to first choose the most specific or the closest CNG next to the affected gene with the epigenetic chagnes. This CNG is then tied to the corrected gene with the right epigenetic sequences by a linker (the CNG and deDRNA complex) before incorporated into a vehicle of gene transfer or viral vector. The treatment is effected by the delivery of this vehicle with the CNG complex either locally into the affected cell or systemically throughout the body. The vehicle with the CNG complex can also be transfer into a stem cell first with the potential to differentiate into the right target cell type and then delivered locally or systemically.

It should be reminded that the use of any linker between the CNGs and the deDRNAs may not be necessary if a simple mixture or concoction of the two can do the job. The designed DNA and RNA may be in the form of a whole gene, cDNA, EST, with or without the promoter or regulator region etc. The choice can be any or all of the above to those that are skilled in the art.

### EXAMPLE V

CNG complex can also be used to replace the defective gene that can cause disease or cancer with the wild type equivalent. The defective gene or genes can be caused by, and not limited to, mutation, deletion, and translocation etc. These defects can be caused by, and not limited to, inheritance, radiation, toxins, and carcinogens etc. As illustrated in example IV, the treatment using the CNG complex can be achieved locally by site injection and infusion with or without vectors or be achieved systemically by infusion with the appropriate cell type. The routes of administration are well known to those skilled in the art.

In another embodiment, the CNG complex can be used to not only replace the defective gene with the wild type, but with a different gene that may even be better in the control or treatment of the disease than the wild type.

Once again, the construction of the CNG complex is to first choose the most specific or the closest CNG next to the affected gene. This CNG is then tied to the corrected or redesigned gene by a linker (the CNG and deDRNA complex) before incorporated into a vehicle of gene transfer or viral vector. The treatment is effected by the delivery of this vehicle with the CNG complex either locally into the affected cell or systemically throughout the body. The vehicle with the CNG complex can also be transfer into a stem cell first with the potential to differentiate into the right target cell type and then delivered locally or systemically.

### EXAMPLE VI

The construction of the CNG complex as described and the methods of delivery can also be applied to germ cell so that certain inheritable diseases can be eliminated in the next or any future generations to come. This can be applied to plants and animals for a better agricultural yield. It may also be applicable for the most serious inheritable diseases in human. It will, of course, be able to survive the medical ethical issues in such a manipulation.

### WORKING EXAMPLE FOR THE DETECTION OF CANCER

A working example of using this invention for the detection of cancer in humans was illustrated by the use of CNGs related to the telomerase gene.

In this aspect, the present invention features methods for the general screening of cancers by determining the amount of a chosen CNG sequence close to the telomerase gene (TECNG) present in the serum or plasma of such patients. Accordingly, the present invention does have broad applicability in clinical medicine.

The methods according to the present invention generally comprise the steps of (1) obtaining a blood sample from a patient, (2) extracting DNA from the blood sample, (3) measuring the amount of circulating TECNG present in the blood sample, and (4) comparing the amount of circulating TECNG present in the blood sample to a control.

Preferably, the blood sample is a non-cellular fluid sample. By non-cellular we mean that the sample is either blood sera where the cells are extracted by clotting and separation of the cells from the remaining fluid or by inhibiting clotting and centrifuging the fluid fraction (plasma). The TECNG is measured from the fluid fraction.

### Materials and Methods

Thirty one patients with known cases of cancer (group A) and fifty five presumably normal individuals (group B) had sent in their blood samples for circulating TECNG determination in our laboratory Hong Kong.

DNA Extraction from Plasma Samples: Peripheral blood (5 ml) was collected from each subject into an EDTA tube for the isolation of plasma. Blood samples were centrifuged at 1600× g, and plasma carefully removed from the EDTA-containing tubes and transferred into plain polypropylene tubes. The samples were stored at −20° C. until further processing. DNA form plasma samples were extracted using a QIAamp Blood Kit (Qiagen, Hilden, Germany) using the blood and body fluid protocol as recommended by the manufacturer (2). Plasma samples (130-800 μl/column) were used for DNA extraction. The exact amount was documented for the calculation of the target DNA concentration. A final elution volume of 50 μl was used from the extraction columns.

More specifically, a real-time quantitative PCR systems had been developed for TECNG detection: The amplification primers consisted of:

forward primer (SEQ ID NO: 1) Tert-1-F
(5′-CAGCGTCAGGAAATAAATGACACA-3′),
reverse primer (SEQ ID NO: 2) Tert-1-R
(5′-ATAACAACAGAAGACAGAGGCTACTTTG-3′)
and the
dual-labeled fluorescent probe (SEQ ID NO: 3)
Tert-1-Probe
[5′-(6-FAM)TCCTGCCTCCAGCTTCCTGGCCAG(TAMRA)-3′].

Fluorogenic PCR reactions were set up in a reaction volume of 50 μl using components (except for the fluorescent probes and amplification primers) supplied in a TaqMan PCR Core Reagent Kit (Perkin-Elmer Corp.). Fluorescent probes were custom-synthesized by Perkin-Elmer Applied Biosystems. Each reaction contained 5 μl of 10× buffer A; 300 nM of each of the amplification primers; 25 nM (for the TECNG probes) 4 MM MgCl₂; 200 μm each of dATP, dCTP, and dGTP; 400 μM dUTP; 1.25 units of AmpliTaq Gold; and 0.5 unit of AmpErase uracil N-glycosylase.

DNA amplifications were carried out in a 384-well reaction plate format in a Applied Biosystems 7900 Sequence Detector. Each sample was analyzed in duplicate. Multiple negative water blanks were included in every analysis.

A calibration curve was run in parallel and in duplicate with each analysis, using DNA synthesized by a commercial clonal expression system as a standard. Concentrations of circulating cell-free TECNG were expressed as copies of TECNG/ml plasma.

Thermal cycling was initiated with a 2-min incubation at 50° C. for the uracil N-glycosylase to act, followed by an initial denaturation step of 10 min at 95° C., and then 40 cycles of 95° C. for 15 s and 56° C. for 1 min were carried out.

Amplification data collected by the 7900 Sequence Detector was then analyzed using the Sequence Detection System software developed by Perkin-Elmer Applied Biosystems. The mean quantity of each duplicate wass used for further concentration calculation. The plasma concentration of TECNG is calculated as followed.

$$C = Qx \begin{matrix}{V}_{DNA} \\ {V}_{pcr}\end{matrix} x \begin{matrix}1 \\ {V}_{ext}\end{matrix}$$

in which C represents the target concentration in plasma (copies/ml), Q represents the target quantity (copies) determined by a sequence detector in a PCR, V_{DNA} represents the total volume of DNA obtained after extraction (typically 50 μl/Qiagen extraction), V_{PCR} represents the volume of DNA solution used for PCR (typically 5 μl, and Vₑₓₜ represents the volume of plasma/serum extracted (typically 0.13-0.80 ml)).

### Results

There was a significant increase of the quantity of TECNG levels in those individuals that have known cases of cancer as compared with those that are negative. This significant increase of TECNG provided evidence that the concept that is behind this invention is sound, as the telomerase gene is activated in most cancerous tissues, whereas it is not in normal ones. The specially conserved non gene sequences of this gene will circulate in the peripheral blood in these patients as suggested by this invention in far higher quantities than that of the normal individuals. The detection of which can be used as a screening or diagnostic test for cancer.

Sequence ID Listing

Forward primer: Tert-1-F

(SEQ ID NO: 1)

(5′-CAGCGTCAGGAAATAAATGACACA-3′),
Reverse primer: Tert-1-R

(SEQ ID NO: 2)

(5′-ATAACAACAGAAGACAGAGGCTACTTTG-3′)
Dual-labeled fluorescent probe: Tert-1-Probe

(SEQ ID NO: 3)

[5′-(6-FAM)TCCTGCCTCCAGCTTCCTGGCCAG(TAMRA)-3′].

1. A method to use the study of conserved non-genic sequences (CNGs) to detect the presence of abnormal changes in genetic or epigenetic functions of any one gene or a group of genes working synergistically that cause diseases by examining the presence or quantification of CNGs:
a. In any biological samples, the intact or close to intact DNA or RNA sequences that are related to that particular gene
b. In any biological sample, the disintegrated forms of DNA or RNA sequences (diDRNAs) that are related to that particular gene

2. A method of claim 1 in which the there may be one or more of CNGs for any one particular gene with genetic or epigenetic changes in any one disease.

3. A method of claim 1 in which the CNGs can be located in either one or in any other numbers of chromosomes in which the location may or may not be physically close to the diseased gene for study.

4. A method of claim 1 in which the CNGs can be located close to, directly linked up with, or within the diseased gene for study.

5. A method of claim 1 in which the detection of the CNGs and the genetic and epigenetic changes can be done by, but not limited to, PCR and any method or methods known to those skilled in the arts.

6. A method of claim 1 in which the detection of the CNGs can be done with any biological samples such as, but not limited to, blood and other bodily fluids.

7. A method of claim 1 in which the detection of the CNGs can be done on a biological sample in one individual while the detection is for the disease or condition of another individual, such as the example of fetal DNA being found in the maternal blood.

8. A method of using conserved non-genic sequences (CNGs) together with the designed or corrected genetic or epigenetic sequences or RNA sequences (deDRNAs) for the alteration of bodily functions or the treatment of diseases by local incorporation or system administration so that the delivery will be effective to the target cell.

9. A method of claim 8 in which the target is in one locus in one chromosome for each disease or condition.

10. A method of claim 8 in which the targets are in multiple loci in one or more chromosomes for each disease or condition.

11. A method of claim 8 in which the target cell is a plant cell.

12. A method of claim 8 in which the target cell is an animal cell.

13. A method of claim 8 in which the target cell is a human cell.

14. A method of claim 8 in which the target cell is a stem cell.

15. A method of claim 8 in which the target cell is a germ cell.

16. A method of claim 8 in which the CNGs and deDRNAs are separately delivered or separately bound to the vehicle or vector before delivery to the target cell.

17. A method of claim 8 in which the CNGs and deDRNAs are linked chemically or biologically first before delivery or bound to the vehicle or vector before delivery to the target cell.

18. A method of claim 8 in which the CNGs and deDRNAs are first created or produced in a cell before delivery into the host organism to be effective in another cell.

19. A method of claim 8 in which the delivery of the CNGs and deDRNAs is done by or with the use of different chemicals, liposomes, non-liposomes, dendrimers, electropolation, gene gun, viral, microinjections etc and other methodologies known to those skilled in the art.
